(19) United States
(12) Patent Application Publication
(10) Pub. No.: US 2008/0268109 A1
(43) Pub. Date: Oct. 30, 2008

(54) Low glycemic sweetener compositions

(76) Inventors: Stephen B. Roman, Phoenix, AZ (US); Gary S. Kehoe, Phoenix, AZ (US)

Correspondence Address: TOD R NISSLE, PO BOX 55630, PHOENIX, AZ, 85078, US

(21) Appl. No.: 11/975,588

(22) Filed: Oct. 18, 2007

Related U.S. Application Data
(63) Continuation-in-part of application No. 11/900,783, filed on Sep. 12, 2007.
(60) Provisional application No. 60/852,945, filed on Oct. 19, 2006.
(60) Provisional application No. 60/844,004, filed on Sep. 12, 2006.

(51) Int. Cl. G01N 33/02

(52) U.S. Cl. 426/231

(57) ABSTRACT

A method is provided to sweeten a food product to minimize the glycemic index of the food product, to introduce natural fiber into the food product, to produce a desired sweetness profile, and to reduce the likelihood that the food product when ingested functions as a laxative. The method includes the steps of selecting at least one polyol; determining the Taxation threshhold in grams per day of the polyol; determining the desired quantity of a sweetener composition in a serving of the food product, the sweetener composition comprising the polyol and agave syrup solids added in solid form during the manufacture of the food product; determining the desired sweetener profile of the sweetener composition, the sweetener composition including proportions of agave and the polyol to minimize the likelihood the sweetener composition will cause the food product to function as a laxative; admixing the polyol and agave syrup to produce a sweetener mixture; drying and fractioning the sweetener mixture to produce solid sweetener particles infused with agave syrup solids; and, admixing the sweetener particles during the manufacture of the food product to produce the desired quantity of the sweetener composition in a serving of the food product.

This application claims priority based on provisional application Ser. No. 60/852,945, filed Oct. 19, 2006, and is a continuation-in-part of patent application Ser. No. 11/900,783, filed Sep. 12, 2007, and its associated provisional application Ser. No. 60/994,004, filed Sep. 12, 2006.

This invention relates to sweetener compositions.

More particularly, the invention relates to sweetener compositions that minimize the glycemic index of a food product, that introduce natural fiber into the food product, that produce a desired sweetness profile, and that reduce the likelihood that the food product when ingested functions as a laxative.

In a further respect, the invention relates to sweetener compositions that simultaneously release from a food product a polyol and agave syrup when the food product is in the digestive tract of an individual.

The use of polyols in food products in combination with various sweeteners is well known. The sweeteners are added to an already manufactured polyol, or are added during the manufacture of a food product as an ingredient separate from the polyol. Polyols have a lower glycermic index than sucrose or high fructose corn syrup, and, as do a variety of other substances, have a Taxation threshold.

We have discovered a sweetener composition and method that enables, simply by adding the sweetener composition during manufacture of the product a food product, the achievement of a desired sweetness profile in the food product for a polyol and associated sweetener, of a desired glycemic index, of a desired laxative effect, and of the simultaneous release of a polyol and associated sweetener in the digestive tract of an individual that consumes one or more servings of the food product.

The method includes the steps of selecting at least one polyol; determining the Taxation threshold in grams per day of the polyol; determining the desired quantity of a sweetener composition in a serving of the food product, the sweetener composition comprising the polyol and agave added in solid or liquid form during the manufacture of the food product; determining the desired sweetener profile of the sweetener composition, the sweetener composition including proportions of agave and the polyol to minimize the likelihood the sweetener composition will cause the food product to function as a laxative; admixing the polyol and agave in liquid form to produce a sweetener mixture; drying and fractioning the sweetener mixture to produce solid sweetener particles infused with agave; and, admixing the sweetener particles during the manufacture of the food product to produce the desired quantity of the sweetener composition in a serving of the food product. A minor selected amount of a high intensity sweetener can be admixed with the polyol and agave syrup. Prior to admixing the sweetener particles during the manufacture of the food product, the sweetener particles can be transported as a solid bulk sweetener-polyol composition to the site at which the food product is manufactured.

The polyol selected can, by way of example and not limitation, comprise sorbitol, mannitol, polyglycitol syrup, polyglycitol powder, maltitol syrup, maltitol, xylitol, lactitol monohydrate, anhydrous isomalt, and erythritol. The glycemic index of these polyols is low in comparison to sucrose. The laxation threshold in grams/day of these polyols is sorbitol (50), mannitol (20), polyglycitol syrup (greater than 100), polyglycitol powder (greater than 150), maltitol syrup (greater than 100), malititol (60-90) xylitol (50-90), lactitol monohydrate (20-50), anhydrous isomalt (50-70), and erythritol (125). The % relative sweetness of these polyols with respect to sugar is sorbitol (60), mannitol (50), polyglycitol syrup (30-40), polyglycitol powder (less than 20), maltitol syrup (70-80), malititol (90) xylitol (100), lactitol monohydrate (30-40), anhydrous isomalt (40), and erythritol (60-70). Agave syrup is 120% as sweet as sucrose. In addition, the Taxation threshold of agave syrup is less than most, if not all, polyols. Further, in one test premium agave syrup had a palatability of about 45 mm and a glycemic index of about 33.

In an alternate embodiment of the invention, a method is provided to sweeten a food product to minimize the glycemic index of the food product, to introduce natural fiber into the food product, to produce a desired sweetness profile, and to reduce the likelihood that the food product when ingested functions as a laxative. The method comprises the steps of selecting at least one polyol; determining the Taxation threshhold in grams per day of the polyol; determining the desired quantity of a sweetener composition in a serving of the food product, the sweetener composition comprising the polyol and agave syrup added in liquid form during the manufacture of the food product; determining the desired sweetener profile of the sweetener composition, the sweetener composition including proportions of agave and said polyol to minimize the likelihood the sweetener composition will cause the food product to function as a laxative; admixing the polyol and agave syrup to produce a liquid sweetener mixture infused with agave syrup; and, admixing the liquid sweetener mixture during the manufacture of the food product to produce the desired quantity of the sweetener composition in a serving of the food product. A minor selected amount of a high intensity sweetener can be admixed with the polyol and agave syrup. Prior to admixing the liquid sweetener mixture during the manufacture of the food product, the liquid sweetener can be transported as a liquid bulk sweetener-polyol composition to the site at which the food product is manufactured. And, if desired, both liquid and sold forms of sweetener infused polyol can be used during the manufacture of the food product to produce the desired quantity of the sweetener composition in a serving of the food product.

The desired quantity of sweetener in a food product will, of course, depend on the food product. A desert-type food product like cake or an apple turnover will require a higher weight percent of a sweetener than a “less sweet” food product like bread. Regardless of the food product, however, the quantity of sweetener in a serving of the food product can be readily determined. This is evidenced by the fact that on most food products the serving size and quantity of sugar or other sweeteners is typically set forth on a label on the food product. The desired amount of sucrose or other sweetener utilized in the product is also readily determined either by past experience in making the produce or by current formulation of a desired product. If, for example, fifty grams of sucrose is required in a food product, then the desired quantity of a polyol and an associated sweetener to provide an equivalent sweetness is determined by considering the serving size, by considering the laxation threshold and % relative sweetness of the polyol, and by considering the Taxation threshold and % relative sweetness of the sweetener associated with the polyol.

First, for sake of discussion, assume that the polyol selected is just as sweet as sucrose. Then, if for example the polyol is sorbitol and the sweetener is agave syrup, the laxation threshold of sorbitol is 50 grams/day. If the serving size is one, i.e., the food product likely will be entirely consumed in one sitting, then fifty grams of sorbitol is not utilized in the product because it is likely the product would have a laxative effect. Less than fifty grams of sorbitol is utilized in the product. For example, thirty grams of sorbitol and twenty grams of agave syrup are utilized to insure that the food product likely will not have a laxative effect. If, on the other hand, the food product includes ten servings and it is unlikely that more than a couple servings will be consumed in a day, then fifty grams of sorbitol likely could be used because only five grams of sorbitol is consumed in each serving. As a general rule, it might well be preferred to limit the amount of sorbitol to eight to ten grams per serving to reduce the risk that sufficient sorbitol will be ingested to cause Taxation, although such limitations can be set as desired.

Second, for sake of further discussion, it is noted that sorbitol is only 60% as sweet as sucrose. If the sweetness provided by 50 grams of sucrose is desired in the food product, either more than fifty grams of sorbitol has to be utilized, or the sorbitol has to be combined with or replaced in part by a polyol or sweetener that has a greater % relative sweetness than does sorbitol. Consequently, a high intensity sweetener like Aspartame™ can be utilized, as can agave syrup which typically is 120% as sweet as sugar. Agave syrup is a preferred sweetener in the practice of the invention because it includes soluble inulin fiber.

A food product like gum may well be able to utilize a sweetener that is 100% by weight sorbitol because the serving size is small. Other food products like cakes (or the cake mix used to make the cake), or ice cream are eaten in larger serving sizes which will include a greater quantity of sweetener and which may well, to avoid a Taxation effect, require that other sweeteners be utilized in combination with the polyol.

In sum, as noted in the discussion above, after a desired quantity of sucrose (or another sweetener) in a food product and/or a serving of a food product is determined, a determination is made of the proportion of agave syrup (or other sweetener) or agave syrup solids and polyol necessary to provide an equivalent sweetness and to minimize the likelihood that the polyol in the food product will produce a laxation effect when the food product is consumed. By way of example, and not limitation, in one case where a serving of a food product includes a greater amount of sweetener, the desired sweetener may be 60% by weight sorbitol polyol and 40% by weight agave syrup or agave syrup solids. In another case, where a serving of a food product includes a smaller amount sweetener, the desired sweetener might be 90% by weight sorbitol polyol and 10% by weight agave syrup or agave syrup solids.

In accordance with the invention, the desired sweetener is produced by infusing the polyol with agave syrup. This is accomplished by admixing liquid polyol and liquid agave syrup to produce a sweetener mixture. This mixture can be dried and fractioned to produce sweetener particles. The proportion of sorbitol and agave in the sweetener particles is sufficient to produce a relative sweetness that preferably is, when a weight of sweetener particles equivalent to the weight of sucrose (or of high fructose corn syrup or other sweetener(s)) utilized in a food product, equivalent to within plus or minus five percent of the relative sweetness provided by an equivalent weight of sucrose.

The sweetener particles are transported in bulk to a facility producing the desired food product. When the food product is produced, instead of admixing fifty grams of sucrose, fifty grams of the sweetener particles are admixed. If, however, the relative sweetness of fifty grams of sweetener particles is more or less than the relative sweetness of fifty grams of sucrose, the quantity of sweetener particles utilized can be adjusted to produce a sweetness equivalent to the sweetness of sucrose. During production of the food product, it is preferred that the sweetener particles do not dissolve at all or at least do not completely dissolve. This is important because when the particles do not dissolve, the infused agave syrup solids bound inside the sweetener particles is, along with inulin that comprises part of the agave syrup solids, released simultaneously with the polyol when the particles are dissolved in the mouth, throat, stomach, or intestinal tract of a person who has consumed the food product. Conventional methods of inulin consumption do not allow for this as inulin is either taken as a supplement once per day, or, is added to food at the point of initial consumption and does not mix uniformly in the food stuff and is not bound to the sweeteners in the food stuff. This invention allows for the introduction of a low glycemic fiber portion at a desired time(s) and place(s) during metabolism.

### EXAMPLE I

The following ingredients are provided:

| | |
|---|---|
| Maltitol liquid (or other polyol(s)) | 40% (w/w) |
| Agave syrup (or other sweetener(s)) | 59.10% (w/w) |
| Water | 0.75% (w/w) |
| Lo han guo, high intensity sweetener | 0.15% (w/w) |

Solid agave-infused polyol sweetener particles are produced as follows:
(a) The water is added to a mixing vessel.
(b) The maltitol is admixed with the water to produce a water—maltitol composition.
(c) The agave syrup, and then the lohan guo, is admixed with the water—maltitol composition to produce a sweetener composition. Further desired additives and ingredients may, if desired, be added at this stage and admixed to produce a uniform composition.
(d) The sweetener composition is heated to 330 degrees F. and subjected to a vacuum of 24-27 in/Hg while under agitation. Further desired additives and ingredients may be added at this stage and admixed to produce a more uniform composition.
(e) The heat and vacuum of (d) are maintained until the moisture level in the sweetener composition is below 0.75% by weight.
(f) The cooked mass is aerated to improve appearance and fracturing during subsequent milling.
(g) The cooked mass is formed into a rope via an extrusion process and the rope is stamped into three-quarters of an inch by three-quarters of an inch pillows. Further desired additives and ingredients may, if be added prior or just after the stamping process.
(h) The pillows are cooled to room temperature.
(i) The pillows and classified using a Fitzmill or other fractioning classifier to yield particles of a desired size. Further desired additives and ingredients may be added during this fractioning/classifying process.

The foregoing process can be carried out by a batch process, a continuation process, or a combination of the two. The order of addition and combination of the ingredients can be varied as desired depending on the types of ingredients being blended and the respective physical characteristics of the ingredients.

Examples of equipment that may be utilized during the Example 1 process include, without limitation, drum crystallization, spray dry, extrusion, fluid bed dryer, dry blend/agglomeration, and block crystallization equipment.

### EXAMPLE 2

The following ingredients are provided:

| | |
|---|---|
| Maltitol solid particulate (or other polyol(s)) | 45% (w/w) |
| Agave syrup (or other sweetener(s)) | 45% (w/w) |
| Water | 9.85% w/w) |
| Stevia (Stevioside/ebaudioside-A) | 0.15% (w/w) |

An agave-infused polyol liquid composition is produced as follows:
(a) The water is added to a mixing vessel.
(b) The maltitol is admixed with the water to produce a water—maltitol composition.
(c) The agave syrup, and then the lohan guo, are admixed to uniformity with the water—maltitol composition to produce a sweetener composition. Further desired additives and ingredients may, if desired, be added at this stage and admixed to produce a uniform composition.

In Examples 1 and 2, other ingredients that may be added include fiber, flavors (like vanilla, for example), aromatics, soluble fiber (like inulin, for example), acids, extracts, fruit concentrates, syrups, minerals (like chromium or zinc, for example), appetite suppressants (like hoodia, for example), honey, lo han guo, thaumatin, neohesperidin, dihydrochalcone, and monatin. Examples of other sweeteners include sucralose and brown rice syrup, among others.

The weight percent of a polyol(s) in the solid or liquid compositions produced in Examples 1 and 2 is in the range of 10% to 80% by weight, as is the weight percent of agave syrup (liquid composition) or agave syrup solids (solid composition). When high intensity sweeteners are utilized in the solid or liquid compositions, it is in at a weight percent in the range of 0.0001% to 3.5% by weight.

In one embodiment of the invention, the sweetener-infused polyol liquid compositions presently include 26% to 75% by weight polyol, 15% to 65% by weight natural sweetener (such as agave syrup), and 5% to 45% by weight water. The liquid compositions can also include 0.0001% to 3.5% by weight of a high potency sweetener such as lo han guo.

In another presently preferred embodiment of the invention, the sweetener-infused polyol liquid compositions presently include 46% to 56% by weight polyol, 44% to 54% by weight natural sweetener (such as agave syrup), and 15% to 25% by weight water. The liquid compositions can also include 0.01% to 1.5% by weight of a high potency sweetener such as lo han guo.

In another embodiment of the invention, the sweetener-infused polyol solid compositions presently include 36% to 85% by weight polyol, 15% to 64% by weight natural sweetener (such as agave syrup solids), and 0% to 5% by weight water. The liquid compositions can also include 0.0001% to 3.5% by weight of a high potency sweetener such as lo han guo.

In a further presently preferred embodiment of the invention, the sweetener-infused polyol solid compositions presently include 56% to 66% by weight polyol, 34% to 44% by weight natural sweetener (such as agave syrup solids), and 0.2% to 1.5% by weight water. The liquid compositions can also include 0.01% to 1.5% by weight of a high potency sweetener such as lo han guo.

Any desired polyol(s) can be utilized in producing the sweetener-infused polyol solid and liquid compositions of the invention. Examples of polyols includes, without limitation, maltitol, sorbitol, xylitol, erythritol, lactitol, mannitol, SORBOGEM™ and SORBO® by SPI Polyols, NEOSORB™ by Roquette, SORBITOL SOLUTION™ and CRYSTALLINE SORBITOL™ by Archer Daniels Midland (ADM), SORBODEX™ by Merck & Co. Inc., MALTISWEET™, STAQBILITE™ and HYSTAR® by SPI Polyols, MALTISORB® and LYCASIN® by Roquette, MALTIDEX™ and MALBIT™ by Cerestar, LESYS-T® and AMALTY® MR by Towa Chemical Industry Col, Ltd, and POLYDEXTROSE® by Danisco.

Agave Syrup may be sourced from one or more, without limitation, of the following companies: Nekutli,k IIDEA, AG Commodities, Sweet Cactus Farms, Blue Agave Syrup, and American Health and Nutrition.

The water utilized in the practice of the invention may be de-ionized water, USP water, de-chlorinated water, mineral water, treated water, or tap water. The preferred water is deionized water.

Stevia Rabudiana may be sourced from one or more, but not limited to the following companies: AIDP, Inc.; Alfa Chem; Ama Nutrasource Inc.; American Ingredients; AnMar Nutrition; AsiaAmerica Int'l, Inc. Draco Natural Products, Falcon Trading Int'l. GCI Nutrients; Functional Foods Corp.; Herb Trade Inc.; Motherland Int'l.; NuNaturals, Inc.; Nutriland Group, Inc.; Nutrichem Resources Co.; NutriScience Innovations LLC; Pharmachem Laboratories, Inc.; Pharmline Inc.; Ria Int'l LLC; San Francisco Herb & Natural Food Co.; Starwest Botanicals; Stauber Performance Ingredients; Strategic Sourcing, Inc.; Stryka Otanics; P.O. Thomas & Co., Inc.; Watson Industries; and, Wright Group.

Lo han guo examples include PURELO™ by Bio Vittoria, MORMORDICA™ by Draco Natural Products, Lo Han Guo concentrate by Amax Nutrasource.

Any source of soluble, insoluble and/or fermentable fiber(s) can be utilized in the compositions of the invention including, by way of example and not limitation, gums, hydrocolloids, mucilages, hemicellulose, cellulose, lignin, psyllium seed husk, beta-glucan, pectins, gum arabic, oat bran, locust bean gum, fructans, xanthan gum, fructooligosaccharides, oligosaccharides, polysaccharides, resistant starches, polydextrose, inulin, arabinogalactose, hydrolyzed guar gum, canthan, alginate, bet-glucans, tragacanth, arabinoxyland, NUTRIOSE by Roquette, and FIBERSOL by Matsutani Chemical Industry Col.

Any natural sweetener(s), appetite suppressant(s), and/or high potency sweetener(s) may be used in the compositions of the invention including, by way of example and not limitation, gymnema sylvesgtre, vanadyl sulfate, bitter melon extract, fenugreek extract, bilberry extract, cinnamon extract, jambolan, pterocarpus marsupium, gulvel, d-tagtose, raw organic honey, maple syrup, inulin, barley malt, lactitol, yacon, brown rice syrup, granulated brown rice sweetener, amasake, fruit concentrate, fruit juice sweeteners, organic molasses, sorphum syrup, date sugar, organic dried natural can juice, trehalose, and carralluma fimbriata.

As used herein, a polyol is a hydrogenated form of carbohydrate. Whose carbonyl group (aldehyde or ketone, reducing sugar) has been reduced to a primary or secondary hydroxyl group.

As used herein, an infused polyol liquid or solid is a polyol in which a sweetener or other component or composition is generally homogeneously dispersed throughout the polyol liquid or solid.

As used herein, a solid polyol particle contains at least 36% to 85% by weight of one or more polyols.

As used herein, a solid polyol particle infused with a sweetener contains 36% to 85% by weight polyol and 15% to 64% by weight sweetener.

As used herein, a sweetener is any natural or synthetic sweetener including, but not limited to, synthetic high intensity sweeteners such as lo han guo, stevia, acesulfame potassium, and sucralose. Agave syrup is an example of a natural low glycemic sweetener.

As used herein, a polyol liquid contains 26% to 75% by weight polyol, 15% to 65% by weight sweetener, and 5% to 45% by weight water.

As used herein, a solid bulk sweetener-polyol composition consists of sweetener-infused solid polyol particle composition shipped in a container holding at least fifty pounds of the solid sweetener infused polyol particle composition.

As used herein, a liquid bulk sweetener-polyol composition is a sweetener-infused liquid polyol composition shipped in a container holding at least forty pounds of the sweetener-infused liquid polyol composition.

Having described our invention in such terms as to enable it to be made and used by those of skill in the art, we Claim:

1. A method to sweeten a food product to minimize the glycemic index of the food product, to introduce natural fiber into the food product, to produce a desired sweetness profile, and to reduce the likelihood that the food product when ingested functions as a laxative, said method comprising the steps of
(a) selecting at least one polyol;
(b) determining the Taxation threshhold in grams per day of said polyol;
(c) determining the desired quantity of a sweetener composition in a serving of the food product, said sweetener composition comprising said polyol and agave syrup solids added in solid form during the manufacture of the food product;
(d) determining the desired sweetener profile of said sweetener composition, said sweetener composition including proportions of agave and said polyol to minimize the likelihood said sweetener composition will cause the food product to function as a laxative;
(e) admixing said polyol and agave syrup to produce a sweetener mixture;
(f) drying and fractioning said sweetener mixture to produce solid sweetener particles infused with agave syrup solids;
(g) admixing said sweetener particles during the manufacture of the food product to produce said desired quantity of said sweetener composition in a serving of the food product.

2. A method to sweeten a food product to minimize the glycemic index of the food product, to introduce natural fiber into the food product, to produce a desired sweetness profile, and to reduce the likelihood that the food product when ingested functions as a laxative, said method comprising the steps of
(a) selecting at least one polyol;
(b) determining the Taxation threshhold in grams per day of said polyol;
(c) determining the desired quantity of a sweetener composition in a serving of the food product, said sweetener composition comprising said polyol and agave syrup added in liquid form during the manufacture of the food product;
(d) determining the desired sweetener profile of said sweetener composition, said sweetener composition including proportions of agave and said polyol to minimize the likelihood said sweetener composition will cause the food product to function as a laxative;
(e) admixing said polyol and agave syrup to produce a liquid sweetener mixture infused with agave syrup;
(f) admixing said liquid sweetener mixture during the manufacture of the food product to produce said desired quantity of said sweetener composition in a serving of the food product.

3. The method of claim 1 wherein in step (e) a minor selected amount of a high intensity sweetener is admixed with said polyol and agave syrup.

4. The method of claim 2 where in step (e) a minor selected amount of a high intensity sweetener is admixed with said polyol and agave syrup.
